(19) United States
(12) Patent Application Publication
(10) Pub. No.: US 2007/0260041 A1
(43) Pub. Date: Nov. 8, 2007

(54) Allylic disulfide rearrangement and desulfurization method for the preparation of allylic sulfides

(76) Inventors: David Crich, Chicago, IL (US); Franck Brebion, Paris (FR); Venkataramanan Krishnamurthy, Plainsboro, NJ (US)

Correspondence Address: OLSON & HIERL, LTD., 20 NORTH WACKER DRIVE, 36TH FLOOR, CHICAGO, IL, 60606, US

(21) Appl. No.: 11/799,247

(22) Filed: May 1, 2007

Related U.S. Application Data
(60) Provisional application No. 60/797,126, filed on May 3, 2006.
(60) Provisional application No. 60/830,761, filed on Jul. 13, 2006.

(51) Int. Cl. C07K 1/02; C07H 15/00

(52) U.S. Cl. 530/333; 530/410; 536/4.1; 562/557

(57) ABSTRACT

A method for the preparation of an allylic sulfide comprises contacting an activated sulfenyl compound of Formula (I) with a thiol of Formula (II) for a period of time sufficient to form an intermediate of Formula (III), and contacting the intermediate of Formula (III) with a thiophilic agent, in a polar solvent, to induce a [2,3]-sigmatropic rearrangement therein and thereby form an allylic sulfide of Formula (IV), with concomitant loss of a sulfur atom to the thiophilic agent, wherein in Formulas (I, II, II, and IV), X is S or SO₂; Y is an aryl group, a substituted-aryl group, a heteroaryl group , or a substituted heteroaryl group; R¹, R², R³, R⁴, and R⁵ are each independently H, a hydrocarbon moiety or a substituted hydrocarbon moiety; and R is an organic moiety.

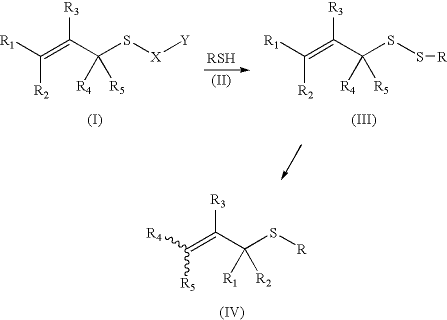

### CROSS-REFERENCE TO RELATED APPLICATIONS

This application claims the benefit of Provisional Application for Patent Ser. No. 60/797,126, filed on May 3, 2006, and Provisional Application for Patent Ser. No. 60/830,761, filed on Jul. 13, 2006, each of which is incorporated herein by reference in its entirety.

### STATEMENT OF GOVERNMENT INTEREST

A portion of the work described herein was supported by a government-sponsored grant from the National Institutes of Health, Grant No. GM 62160. The United States government has certain rights in this invention.

### FIELD OF THE INVENTION

The present invention relates to methods of preparing allylic sulfides. More particularly, the present invention relates to methods of preparing allylic sulfides utilizing a [2,3]-sigmatropic rearrangement and desulfurization of a disulfide compound.

### BACKGROUND OF THE INVENTION

The development of methods for the functionalization of biopolymers, especially peptides (e.g., polypeptides and proteins), under the mildest possible conditions (dubbed “ligation”) is a current frontier in organic chemistry. If such methods are to be fully useful and applicable to biochemical and biological systems, then very high chemoselectivity, stability, and compatibility with protic solvents, and preferably aqueous media, are highly desirable.

Thiols, and in particular cysteine residues, have proven popular targets for chemoselective ligation of complex molecules such as peptides. So-called “Native Chemical Ligation” for the synthesis of peptides and proteins, and its enzyme-promoted biochemical equivalent, “Expressed Protein Ligation”, are such reactions, which use the sulfhydryl (i.e., thiol) group to great advantage in the highly chemoselective formation of amide bonds in aqueous solution. Another thiol-based method, the selective formation of mixed disulfides, is both one of the oldest and most enduring of ligation methods. The mildness of the disulfide ligation and its established chemoselectivity for the cysteine thiol in the presence of all the proteinogenic amino acids stands in stark contrast to the various other methods for cysteine functionalization, most of which involve the capture of the cysteine thiol by electrophilic species, and which consequently have obvious potential chemoselectivity issues. The practicality of the disulfide ligation, with its direct applicability to cysteine-containing peptides, also contrasts with the various ingenious indirect methods that have been developed for the preparation of S-functionalized cysteine derivatives, including, for example, the Michael addition of thiols to dehydroalanine units, the alkylation of thiolates with peptide-based β-halo-alanine units, and other electrophiles, the opening of peptide-based aziridines by thiolates, and the synthesis of peptides with previously functionalized cysteine building blocks, each of which requires the synthesis of modified peptides. The many advantages of the disulfide ligation are offset, however, by its impermanence, which results from the lability of the disulfide bond in the presence of thiols and other reducing agents.

Consideration of the practical advantages of the disulfide ligation, and the disadvantages of its impermanence, led us to investigate methods for developing a more permanent sulfhydryl ligation method. In this regard, we previously found that the allylic selenosulfides of complex molecules, such as peptides and carbohydrates, can undergo efficient dechalocogenative rearrangement to allylic sulfides under appropriate conditions (See e.g., Crich, D.; Krishnamurthy, V.; Hutton, T. K. J. Am. Chem. Soc. 2006,128, 2544-2545; and Crich, D.; Brebion, F.; Krishnamurthy, V. Org. Lett. 2006, 8, 3593-3596).

The selenosulfide chemistry involves the deselenative [2,3]-sigmatropic rearrangement of allylic selenosulfides (Scheme 1), and is best suited for the synthesis of tertiary allylic sulfides from primary selenosulfides, as exemplified by the introduction of linalyl and nerolydyl groups.

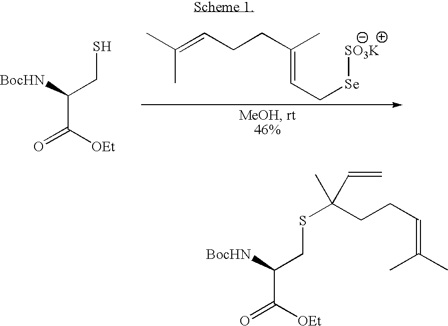

However, this method is not as well adapted for the preparation of primary allyl sulfides due to complications in the synthesis of the required tertiary selenosulfides. In order to access primary allylic sulfides from thiols, we have now examined and report on the analogous rearrangement of allylic disulfides.

As first described by Baldwin, allylic disulfides are in equilibrium with allylic thiosulfoxides by virtue of a [2,3]-sigmatropic rearrangement of undetermined stereoselectivity as shown in Scheme 2 (See Höfle, G.; Baldwin, J. E. J. Am. Chem. Soc. 1971, 93, 6307). The equilibrium, which strongly favors the allylic disulfide, can be displaced toward the formation of an allylic sulfide by the addition of a thiophilic agent. At 60° C. in benzene, rate constants for the rearrangement with transfer of sulfur to triphenylphosphine were found by Baldwin to be strongly dependent on the substitution pattern. Whereas the desulfurative rearrangement was reported to be slow for primary allylic disulfides (R₁,R₂═H and R₃,R₄═H/alkyl; k=0.7-8.6 10⁻⁴ s⁻¹), a significant rate acceleration was observed with secondary and tertiary allylic disulfides (R₁,R₂=alkyl and R₃,R₄═H; k=1.4-1.9 10⁻² s⁻¹).

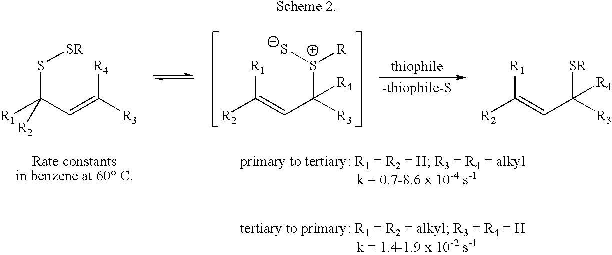

The basis of the present invention is our discovery that the use of polar solvents, which may stabilize the polar thiosulfoxide intermediate, enables the [2,3]-sigmatropic rearrangement reaction and desulfization to be conducted at room temperature, thereby providing a mild and selective functionalization method for thiols, particularly suitable for ligation of complex thiol molecules such as thio-substituted peptides and thio-substituted carbohydrates, and which is complementary to the allylic selenosulfide methodology.

### SUMMARY OF THE INVENTION

The present invention provides a method for the preparation of an allylic sulfide, which is particularly well suited for use with complex molecules such as peptides and carbohydrates. Such allylic sulfides are useful as intermediates in a number of chemical transformations in the preparation of pharmaceuticals and biochemical reagents. The method comprises contacting an activated allylic sulfenyl compound of Formula (I) with a thiol of Formula (II) for a period of time sufficient to form an intermediate of Formula (III), and then contacting the intermediate of Formula (III) with a thiophilic agent (e.g., triphenylphosphine) in a polar solvent (e.g., an alcohol or aqueous buffer) to induce a [2,3]-sigmatropic rearrangement with concomitant loss of a sulfur atom to the thiophile (e.g., as triphenylphosphine sulfide), thereby forming an allylic sulfide of Formula (IV), as set forth in Reaction Scheme (A).

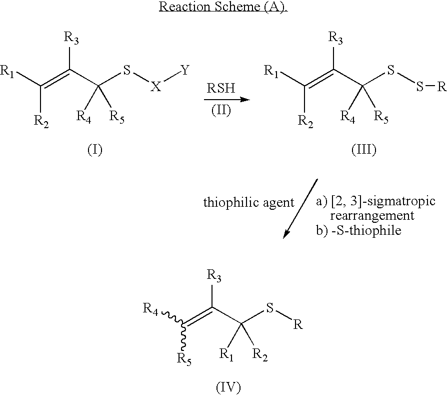

In the formulas set forth in Reaction Scheme (A), X is S or SO₂ (preferably S); Y is an aryl group, a substituted-aryl group, a heteroaryl group (e.g., pyridyl or benzothiazolyl), or a substituted heteroaryl group (e.g., a nitrosubstituted pyridyl); R¹, R², R³, R⁴, and R⁵ are each independently H, a hydrocarbon moiety, or a substituted hydrocarbon moiety; and R is an organic moiety.

The compound of Formula (I) preferably is prepared by sulfenylation of an allylic thiol with a disulfide, YSSY, in a suitable solvent (e.g., chloroform, methanol, acetonitrile, mixtures thereof, and the like). Y is an aryl group, a substituted-aryl group, a heteroaryl group (e.g., pyridyl or benzothiazolyl), or a substituted heteroaryl group (e.g., a nitrosubstituted pyridyl). An amine, such as triethylamine, can be added to accelerate the sulfenylation reaction, if desired. Alternatively, an activated aryl or heteroaryl sulfonate, such as phenylsulfonyl chloride) can be used to prepare compounds of Formula (I) in which X is SO₂.

In some preferred embodiments, Y in Formula (I) is selected from the group consisting of a phenyl group, a substituted-phenyl group, a pyridyl group, a substituted pyridyl group, and a benzothiazolyl group. Preferably, R is an organic moiety selected from the group consisting of an amino acid, a peptide, and a carbohydrate, e.g., derived from a thio-substituted amino acid, peptide or carbohydrate.

In other preferred embodiments, R¹, R², R³, R⁴, and R⁵ are each independently H, an alkyl group, or a substituted alkyl group.

Preferred thiophiles are phosphine reagents, phosphoramides, and the like. Non-limiting examples of suitable phosphine reagents include triphenylphosphine, (4-dimethylaminophenyl)diphenylphosphine (Ph₂P(4-C₆H₄NMe₂)), polymer-bound phosphines (e.g., diphenylphosphenyl-polystyrene), and the like. Polymer-bound phosphines provide a ready means for recovering excess unreacted phosphine, as well as the phosphine sulfide byproduct of the reaction. The recovered polymer-bound phosphine and byproduct can then be recycled and reused.

Hydrocarbon moieties can be saturated or unsaturated, and include, for example, alkyl groups, substituted alkyl groups (e.g., C₁-C₂₀ alkyl), aryl groups (e.g., phenyl, naphthyl, and the like), and substituted-aryl groups, most preferably alkyl groups. Substituted hydrocarbon moieties can be alkyl and/or aryl groups substituted with one or more functional group, including, without limitation, a hydroxyl group, an ether group, an amino group, a carboxyl group, an ester group, an amide group, a carbonyl group, an acetal group, a hemiacetal group, a thiol group, a thioether group, a phosphate group, a phosphate ester group, a halide, a heterocyclic group, a polyethylene oxide group, a fluorinated alkyl group, a peptide group, and the like, as desired.

Preferred organic moieties, are derived from thiol-substituted compounds (RSH), and include, without limitation, alkyl groups (e.g., C₁-C₂₀ alkyl), substituted-alkyl groups, aryl groups, substituted-aryl groups, amino acids, carbohydrates, peptides, nucleic acids, or groups consisting of two or more of the foregoing bound together. Particularly preferred organic moieties are complex molecules such as amino acids, peptides (e.g., polypeptides and proteins), carbohydrates (e.g., sugars and polysaccharides), nucleic acids, and peptide nucleic acids. Amino acids, peptides, and carbohydrates are particularly preferred organic moieties.

Preferably, the thiol of Formula (II) is a thio-substituted amino acid (e.g., cysteine or a cysteine derivative), a thio-substituted peptide (e.g., a cysteine-containing peptide or a derivative thereof), or a thio-substituted carbohydrate.

Any suitable polar solvent can be used in the present invention. Suitable solvents include, without limitation, acetonitrile, tetrahydrofuran, lower alcohols (e.g., C₁-C₃ alcohols such as methanol, ethanol, and isopropanol), water, aqueous buffers, and mixtures thereof.

### BRIEF DESCRIPTION OF THE DRAWINGS

FIG. 1 provides a table of examples illustrating application of the methods of the present invention to thiols, including thio-substituted carbohydrates, thio-substituted amino acids (e.g., cysteine derivatives), and thio-substituted peptides (e.g., cysteine-containing peptides); in the table, notes a-h refer to the following: ^{a}Ar=2-benzothiazolyl. ^{b} The double bond configuration of 13, 18, 22, 23, 25, 26 and 28 was determined by a homonuclear spin decoupling experiment (J˜15 Hz). The E and Z isomers of 21 and 24 were separated by preparative HPLC and double bond configurations were determined on the basis of ¹³C γ effect. ^{c} Reactions were performed in benzene at 0.05 M with 1.3 equiv of disulfide, 3 equiv of phosphine. Et₃N (3 equivalents) was added to accelerate the disulfide bond formation (minutes instead of hours). The disulfide bond formation was carried out at ambient room temperature and the rearrangement at reflux. ^{d} Reactions were performed at ambient room temperature in MeCN/MeOH (1/1) at 0.05 M with 2 equivalents of disulfide and 3 equivalents of phosphine. ^{e} PPh₃ was used as thiophile. ^{f} Ph₂P(4-C₆H₄NMe₂) was used as thiophile. ^{g} Et₃N (5 equiv) was added. ^{h} Reaction was performed at room temperature in a mixture of Tris buffer/MeCN/THF (2/1/1) at 0.02 M with 3 equivalents of disulfide and 5 equivalents of phosphine.

### DETAILED DESCRIPTION OF PREFERRED EMBODIMENTS

The following discussion describes preferred aspects of the present invention, and is not to be construed as limiting the scope thereof.

As used herein and in the appended claims, the term “substituted” as applied to hydrocarbon groups, aryl groups, heteroaryl groups, and other organic groups, means that the group includes one or more functional group such as a hydroxyl group, an ether group, an amino group, a carboxyl group, an ester group, an amide group, a carbonyl group, an acetal group, a hemiacetal group, a thiol group, a thioether group, a phosphonate group, a phosphate group, a phosphate ester group, a phosphoramide group, a halide, a heterocyclic group, and the like, as desired.

As used herein and in the appended claims, the term “alkyl” encompasses saturated hydrocarbon groups, as well as non-aromatic unsaturated hydrocarbon groups, such as alkenyl groups and alkynyl groups. Such alkyl groups can be linear or branched.

As used herein and in the appended claims, the term “peptide” and grammatical variations thereof, refer to compounds including at least two amino acid residues bound together by a peptide (amide) bond, including dipeptides, oligopeptides, polypeptides, proteins, and any derivatives thereof (e.g., peptides including a protecting group, a carbohydrate group, a lipid group, and the like bound to an amino acid residue in the peptide). The amino acid residues in the peptide can be natural proteinogenic L-amino acids, D-amino acids, non-proteinogenic amino acids (e.g., ornithine, β-amino acids, ω-amino acids, and the like), and combinations thereof.

As used herein and in the appended claims, The term “carbohydrate” encompasses sugars, sugar derivatives, polysaccharides, and polysaccharide derivatives. The carbohydrates can include protecting groups (e.g., ethers, esters, and the like) on the hydroxyl groups, or other appended moieties. Carbohydrates also include reduced sugars and polysaccharides, as well as oxidized sugars and polysaccharides.

The term “amino acid” as used herein and in the appended claims encompasses natural proteinogenic L-amino acids and derivatives thereof, as well as non-natural amino acids and derivatives thereof, such as D-amino acids, derivatives of D-amino acids, non-proteinogenic amino acids, and the like.

A particular advantage of the methods of the present invention is that they can be applied to complex molecules such as thio-substituted amino acids (e.g., cysteine), derivatized amino acids (e.g., thio-substituted amino acids bearing protecting groups, such as N-Boc-cysteine), thio-containing peptides (e.g., cysteine-containing peptides), derivatized peptides (e.g., peptides bearing protecting groups), thio-substituted carbohydrates, and derivatized thio-substituted carbohydrates, and the like, under mild conditions (e.g., room temperature).

In order to illustrate the present methods, a series of secondary and tertiary allylic thiols were prepared by exploiting the ease of formation of allylic xanthates and thiocarbamates, and their thermal [2,3]-sigmatropic rearrangement to dithiocarbonates and thiocarbamates (Scheme 3).

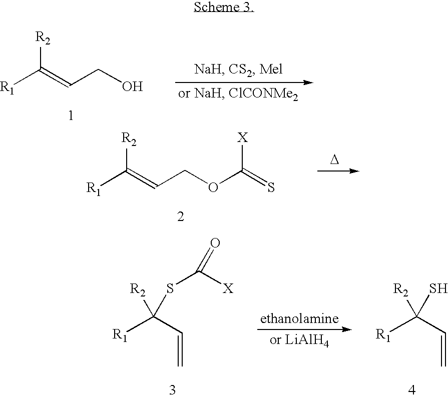

Thiols 4a-c were activated for disulfide formation by conversion to their corresponding benzothiazolyl and pyridyl disulfide derivatives (Scheme 4). Analogous sulfenylating agents have found wide applications for the synthesis of mixed disulfides; especially pyridyl derivatives owing to the spectroscopic properties of the pyridine-4-thiones generated during the reaction, which enable successful monitoring of sulfydryl group modifications. For the preparation of 4a, the benzothiazolyl derivative 5, as well as pyridyl disulfides 6 and 7, were prepared and were easily purified and stable. Selenosulfide 10 was also prepared to compare the efficiency of different sulfenylating agents in the context of this new functionalization method. It is noteworthy that sulfenylating agents derived from hindered allylic thiols have drawn little attention so far and that only a limited number of benzothiazolyl disulfides and thiosulfinates have been reported.

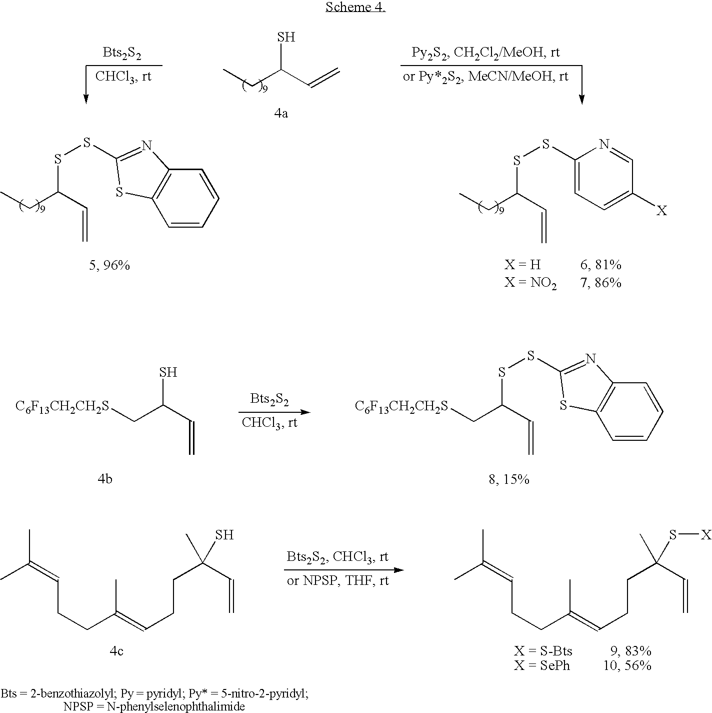

Reaction of disulfide 7 with cysteine derivative 11 at room temperature in CD₃OD/CD₃CN resulted in the formation of the expected disulfide 12 accompanied by only traces of rearranged product 13 (Scheme 5), arising from the [2,3]-rearrangement with loss of sulfur, as determined by NMR. Interestingly, during an attempt to purify 12 on silica gel, a significant amount of 13 was obtained. Sulfur extrusion appears to be favored by the presence of acid in the reaction mixture. In untreated CDCl₃, the disulfide 12 partially rearranged to allylic sulfide 13, even in the absence of phosphine. The addition of a phosphine to a mixture of 12 and 13 resulted in the desulfurization of the thiosulfoxide and led to the primary allylic sulfide as a single E-isomer, without racemization of the cysteine moiety.

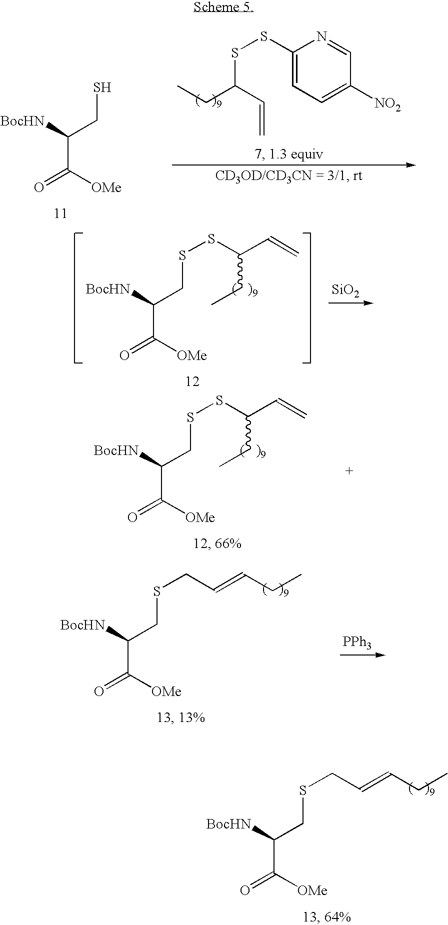

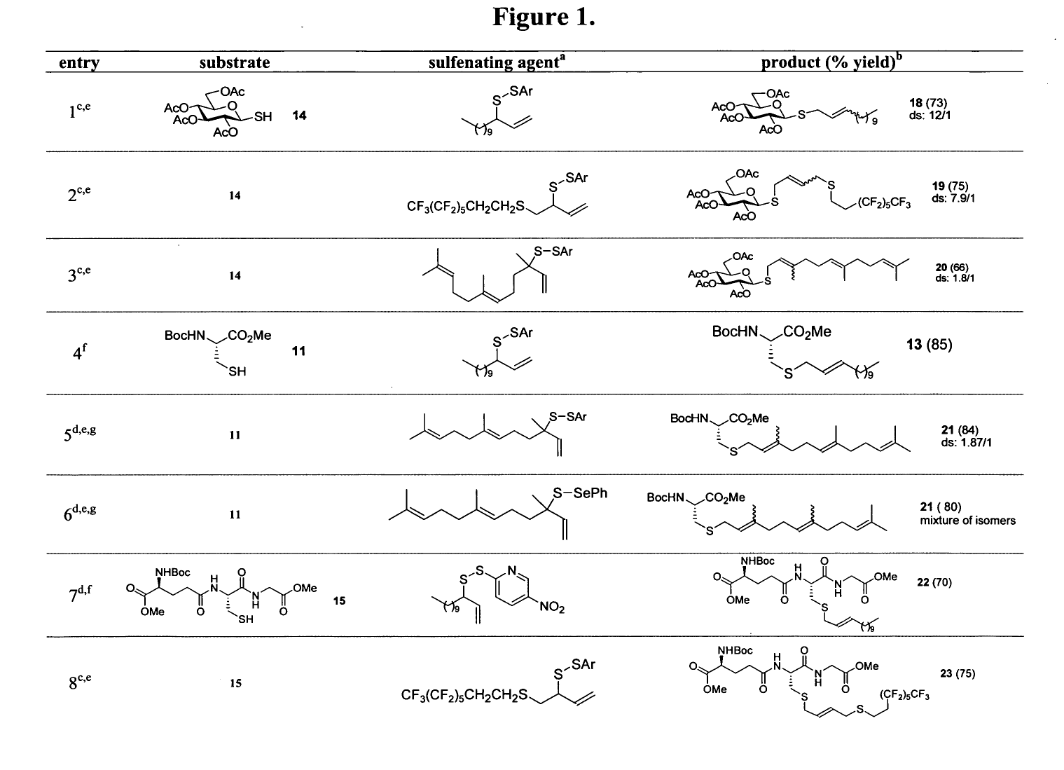

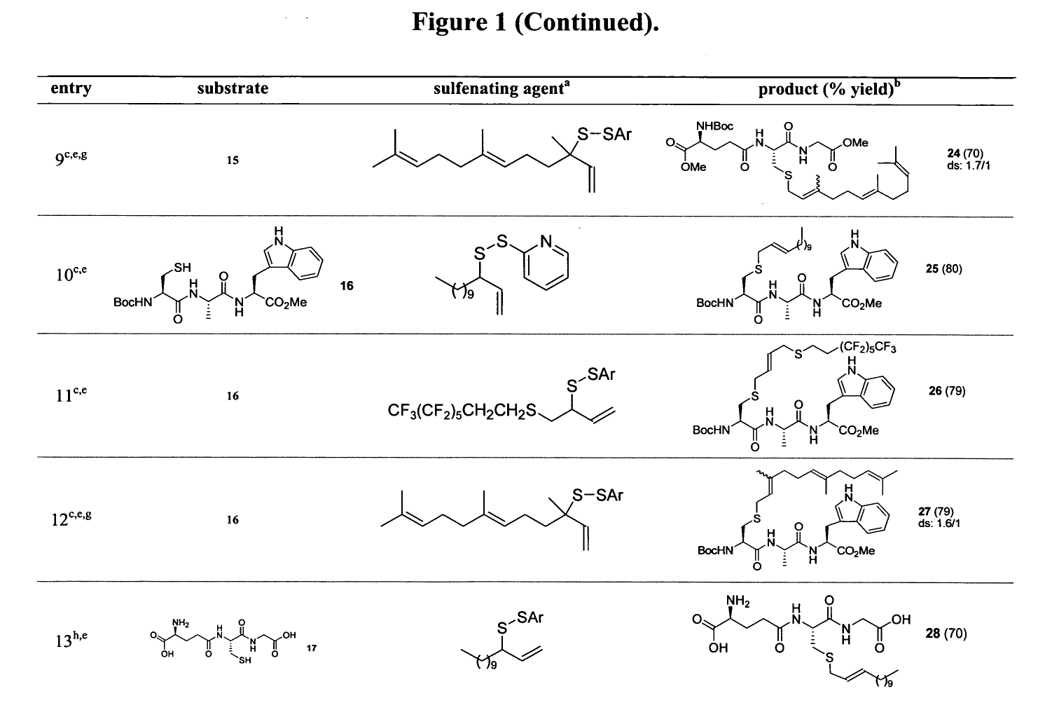

We next explored the possibility of conducting the reaction in one pot, at room temperature, using a mixture of methanol-acetonitrile as solvent. Following this strategy, the phosphine was simply added to the reaction mixture after consumption of the cysteine derivative. This protocol turned out to be not only more convenient, but also more efficient with yields up to 85% (FIG. 1, entry 4). The nature of the sulfenylating agent (5, 6 or 7) or the arylphosphine has no significant impact on the yields, which were optimum with 2 equivalents of sulfenylating agents.

The functionalization of carbohydrate based thiols, cysteine derivatives, and small cysteine-containing peptides was also demonstrated with 1-thio-β-D-glucose tetraacetate 14, Boc-L-Cys-OMe 11, Boc-(α-OMe)-γ-L-Glu-L-Cys-Gly-OMe 15, and Boc-L-Cys-L-Ala-L-Trp-OMe 16 (FIG. 1). These reactions were conducted following the one pot/two step procedure in various solvents, including protic ones. The reaction involving cysteine residues could be performed at room temperature, whereas the rearrangement of disulfides derived from 1-thio-β-D-glucose tetraacetate 14 required higher temperature. Secondary disulfides 5, 6, 7 and 8 were employed under neutral conditions, but the more hindered tertiary disulfide 9, and the selenosulfide 10, required the addition of Et₃N to achieve the ligation to the cysteine derivatives. With selenosulfide 10, the modified cysteine derivative 13 was isolated as a mixture of isomers, presumably arising from double bond isomerization mediated by selenium-based byproducts (FIG. 1, entry 6). The potential of this method for the ligation of organic molecules to cysteine containing peptides was also demonstrated by prenylation of free glutathione (FIG. 1, entry 13). It is noteworthy that this convenient and efficient thiol functionalization method is highly selective and compatible with the indole ring in tryptophan.

The results included in FIG. 1 provide the first insight into the stereochemistry of the [2,3]-sigmatropic rearrangement of allylic disulfides. At room temperature, high selectivity was observed for the formation of E-disubstituted double bonds; but the selectivity slightly decreased when the rearrangement was performed at 80° C. (FIG. 1, entries 1, 2). On the other hand, trisubstituted double bonds were obtained with a modest E-selectivity (FIG. 1, entries 3, 5, 9, 12). These results are consistent with features of other [2,3]-shifts, such as the Evans-Mislow or [2,3]-Wittig sigmatropic rearrangements.

Details of the specific chemical reactions described are provided in the following paragraphs.

General. Unless otherwise stated, ¹H, ¹³C and ¹⁹F NMR spectra were recorded in CDCl₃ solution. Specific rotations were recorded in CHCl₃ solution, unless otherwise stated. All solvents were dried and distilled by standard protocols. All reactions were conducted under a blanket of dry nitrogen or argon. All organic extracts were dried over sodium sulfate, and concentrated under aspirator vacuum at room temperature. Chromatographic purifications were carried out over silica gel. Unless otherwise specified, room temperature is referred to as “rt”; “ether” refers to diethylether (Et₂O); “DMF” refers to dimethylformamide; “EA” refers to ethyl acetate; “AcOH” refers to acetic acid; “Et” refers to ethyl; “Me” refers to methyl; “Ph” refers to phenyl; “min” refers to minutes; “equiv” refers to molar equivalents; “aq” refers to aqueous; “quant” refers to quantitative yield; “mmol” refers to millimoles; “EIHRMS” refers to electron impact high resolution mass spectrum; “ESIHRMS” refers to electron spray ionization high resolution mass spectrum; and “EILRMS” refers to electron impact low resolution mass spectrum.

General procedure for the ligation to 1-thio-β-D-glucose tetraacetate (disulfide bond linkage and [2,3]-sigmatropic rearrangement with extrusion of sulfur). Et₃N (3 equiv) was added to a solution of 1-thio-β-D-glucose tetraacetate (1 equiv) and activated disulfide (1.3 equiv) in benzene (20 mL/mmol of SH) at rt, and the resulting reaction mixture was stirred at rt for 10 minutes. Then triphenylphosphine (3 equiv) was added and the reaction mixture was heated at reflux for 6 hours. The solution was evaporated and the mixture purified by chromatography.

General procedure for the ligation to cysteine derivatives and cysteine containing peptides (disulfide bond linkage and [2,3]-sigmatropic rearrangement with extrusion of sulfur). A solution of cysteine derivative or cysteine containing peptide (1 equiv) and activated disulfide or phenylseleno sulfide (1-3 equiv) in MeOH/acetonitrile: 1/1 (20 mL/mmol of cysteine derivative) was stirred at rt until completion. Then phosphine (2-3 equiv) was added and the reaction mixture was stirred at rt for 10-24 hours. The solution was evaporated and the mixture was purified by chromatography.

(E)-S-Methyl O-tridec-2-enyl carbonodithioate (2a). NaH (7.67 g, 54.0 mmol, 60% in mineral oil) was added in one portion to a solution of trans-2-tridecen-1-ol (10.24 g, 30 mmol) in benzene (120 mL) and THF (20 mL) at rt. The solution was stirred for 1 hour and then carbon disulfide (5.60 mL, 90.0 mmol) was added. After 30 minutes, methyl iodide (3.40 mL, 54.0 mmol) was added slowly and the resulting milky mixture was stirred at rt for 40 minutes. The reaction was quenched with AcOH (3 mL) and filtered. The obtained precipitate was washed twice with Et₂O and the combined organic layers were evaporated. Chromatographic purification (hexane/CH₂Cl₂ from 100/0 to 90/10) afforded 5 (10.38 g, 80%) as a colorless oil. IR (neat): 2923, 2852, 1647, 1464, 1215, 1059 cm⁻¹. ¹H NMR (300 MHz, CDCl₃) δ 5.87 (m, 1H), 5.68 (m, 1H), 5.03 (d, J=6.7 Hz, 2H), 2.56 (s, 3H), 2.07 (q, J=6.9 Hz, 2H), 1.40-1.20 (m, 16H), 0.88 (t, J=6.9 Hz, 3H). EILRMS: calc. for C₁₃H₂₃NOS [M]⁺ 288.2 (1%), 180.2 (78%), 111.1 (56%), 97.1 (95%), 83.1 (100%), 69.1 (97%), 55.1 (97%), 43.1 (80%).

S-Methyl S-tridec-1-en-3-yl carbonodithioate (3a). A solution of 2a (10.3 g, 23.86 mmol) in benzene (120 mL) was heated at reflux for 4 hours. Evaporation of the solvent afforded 3a (10.23 g, quant) as a slightly yellow oil. IR (neat): 3084, 2926, 2854, 1736, 1647, 1464, 920 cm⁻¹. ¹H NMR (400 MHz, CDCl₃) δ 5.75 (m, 1H), 5.24 (dt, J=17.0, 1.0 Hz, 1H), 5.09 (d, J=10.1 Hz, 1H), 4.16 (q, J=7.6 Hz, 1H), 2.40 (s, 3H), 1.68-1.62 (m, 2H), 1.36-1.23 (m, 16H), 0.87 (t, J=6.8 Hz, 3H). ¹³C NMR (100 MHz, CDCl₃) δ 189.3, 137.8, 116.8, 48.8, 34.3, 32.2, 29.9, 29.8, 29.7, 29.6, 29.5, 27.3, 23.0, 14.4, 13.3. EIHRMS: calc. for C₁₅H₂₈OS₂ [M]⁺ 288.15816, found 288.1589.

Tridec-1-ene-3-thiol (4a). A solution of 3a (5.6 g, 19.41 mmol) and ethanolamine (7.1 mL, 116.46 mmol) in THF (40 mL) was stirred at rt for 7 hours and evaporated. Chromatographic purification (hexane) afforded 4a (3.87 g, 93%) as a slightly yellow oil. ¹H NMR (400 MHz, CDCl₃) δ 5.79 (m, 1H), 5.07 (dt, J=16.9, 1.0 Hz, 1H), 4.95 (d, J=9.7 Hz, 1H), 3.41 (m, 1H), 1.69-1.55 (m, 2H), 1.40-1.24 (m, 16H), 0.88 (t, J=6.5 Hz, 3H). ¹³C NMR (100 MHz, CDCl₃) δ 142.3, 114.0, 43.3, 38.1, 32.2, 29.9, 29.8, 29.6, 29.5, 27.7, 23.0, 14.4. EIHRMS: calc. for C₁₃H₂₆S [M]⁺ 214.17552, found 214.17550.

2-(2-(Tridec-1-en-3-yl)disulfanyl)benzo[d]thiazole (5). A solution of 4a (643 mg, 3.0 mmol)) in chloroform (8 mL) was added, dropwise, to a well stirred suspension of 2,2′-dithiobis(benzothiazole) (1.097 g, 3.3 mmol) in chloroform (45 mL). The addition was performed over a period of 15-30 min and the reaction mixture was stirred until completion. The solution was evaporated and disulfide was purified by chromatography (hexane/EA from 100/0 to 95/5) to afford 5 (1.10 g, 96%) as a yellow oil. IR (neat): 3062, 2924, 2852, 1462, 1425, 1005 cm⁻¹. ¹H NMR (400 MHz, CDCl₃) δ 7.85 (d, J=8.1 Hz, 1H), 7.80 (d, J=8.1 Hz, 1H), 7.43 (m, 1H), 7.33 (m, 1H), 5.68 (ddd, J=16.9, 9.7, 9.6 Hz, 1H), 5.18-5.13 (m, 2H), 3.55 (m, 1H), 1.83 (m, 1H), 1.69 (m, 1H), 1.48-1.15 (m, 16H), 0.88 (t, J=6.8 Hz, 3H). ¹³C NMR (100 MHz, CDCl₃) δ 174.0, 155.4, 136.6, 136.1, 126.5, 124.7, 122.4, 121.3, 119.1, 56.0, 36.7, 32.2, 29.9, 29.8, 29.7, 29.6, 29.5, 27.6, 23.0, 14.4. ESIHRMS: calc. for C₂₀H₃₀NS₃ [M+H]⁺ 380.15349, found 380.15331.

2-(2-(Tridec-1-en-3-yl)disulfanyl)pyridine (6). Aldritol (1.17 g, 5.33 mmol) was added in one portion to a solution of thiol 4a (381 mg, 1.77 mmol) in MeOH/CH₂Cl₂: 1/1 (36 mL). The reaction mixture was stirred at rt for 4.5 hours and evaporated. Chromatographic purification (hexane/ether from 100/0 to 97/3) afforded 6 (464 mg, 81%) as a slightly yellow oil. IR (neat): 3045, 2926, 2852, 1574, 1446, 1417, 1119 cm⁻¹. ¹H NMR (400 MHz, CDCl₃) δ 8.42 (d, J=4.6 Hz, 1H), 7.71 (dd, J=8.0 Hz, 1H), 7.61 (m, 1H), 7.05 (m, 1H), 5.62 (ddd, J=16.9, 9.7, 9.5 Hz, 1H), 5.09-5.01 (m, 2H), 3.36 (m, 1H), 1.77 (m, 1H), 1.62 (m, 1H), 1.45-1.10 (m, 16H), 0.87 (t, J=6.7 Hz, 3H). ¹³C NMR (100 MHz, CDCl₃)δ 161.2, 149.5, 137.4, 137.0, 120.6, 120.0, 117.9, 55.3, 33.6, 32.2, 29.8, 29.7, 29.6, 29.55, 29.4, 27.6, 22.9, 14.4. ESIHRMS: calc. for C₁₈H₃₀NS₂ [M+H]⁺ 324.18142, found 324.18121.

5-Nitro-2-(2-(tridec-1-en-3-yl)disulfanyl)pyridine (7). A solution of thiol 4a (590 mg, 2.75 mmol) in MeOH/acetonitrile: 1/1 (20 mL) was added to a suspension 2,2′-dithiobis(5-nitropyridine) (2.56 g, 8.25 mmol) in MeOH (10 mL) and acetonitrile (25 mL) over a period of 15 minutes. The reaction mixture was stirred at rt for 2 hours, filtered through Celite, and evaporated. Chromatographic purification (hexane/ether from 95/5 to 90/10) afforded 7 (880 mg, 86%) as a slightly yellow oil. IR (neat): 2926, 2852, 1587, 1566, 1518, 1342, 1097 cm⁻¹. ¹H NMR (400 MHz, CDCl₃) δ 9.24 (d, J=2.7 Hz, 1H), 8.37 (dd, J=8.9, 2.7 Hz, 1H), 7.90 (d, J=8.9 Hz, 1H), 5.60 (m, 1H), 5.08 (d, J=16.9 Hz, 1H), 5.03 (d, J=10.2 Hz, 1H), 3.42 (m, 1H), 1.76 (m, 1H), 1.45-1.33 (m, 2H), 1.30-1.20 (m, 14H), 0.87 (t, J=6.7 Hz, 3H). ¹³C NMR (100 MHz, CDCl₃) δ 170.0, 145.1, 142.1, 136.9, 131.6, 119.6, 118.7, 55.9, 33.7, 32.2, 29.9, 29.8, 29.7, 29.6, 29.5, 27.6, 23.0, 14.4. EIHRMS: calc. for C₂₀H₃₀NS₃ [M]⁺ 368.15922, found 368.15700.

4-(3,3,4,4,5,5,6,6,7,7,8,8,8-Tridecafluoro-octylsulfanyl)-but-2-en-1-ol (1b). 3,3,4,4,5,5,6,6,7,7,8,8,8-tridecafluoro-1-octanethiol (2.0 g, 5.26 mmol) was added at room temperature, over a period of 5 minutes, to a solution of EtONa (5.78 mmol prepared from 133 mg of Na) in EtOH (6 mL). Next, a solution of (E) 1-chloro-2-butene-4-ol (613 mg, 5.78 mmol) in EtOH (0.5 mL) was added dropwise and the resulting milky solution was heated at 65° C. for 2 hours. The reaction was quenched with aq NH₄Cl, extracted with CH₂Cl₂, washed with brine and dried over Na₂SO₄. Chromatographic purification (hexane/EA from 100/0 to 70/30) afforded 1b (2.2 g, 92%) as a colorless oil. IR (neat): 3352, 3024, 2935, 1872, 1441, 1362, 1219 cm⁻¹. ¹HNMR(400 MHz, CDCl₃) δ 5.80 (m, 1H), 5.62 (m, 1H), 4.22 (dd, J=6.5, 0.9 Hz, 2H), 3.26 (d, J=7.9 Hz, 2H), 2.70 (m, 2H), 2.37 (m, 2H). ¹³C NMR (100 MHz, CDCl₃) δ 132.0, 128.0, 58.4, 32.3 (t, J_{C-F}=21.8 Hz), 28.8, 22.0. ¹⁹F NMR (282 MHz, CDCl₃) δ −8.4, −42.1, −49.5, −50.5, −51.0, −53.8.

Dithiocarbonic acid S-methyl ester O-[4-(3,3,4,4,5,5,6,6,7,7,8,8,8-tridecafluoro-octylsulfanyl)-but-2-enyl]ester (2b). NaH (227 mg, 5.67 mmol, 60% in mineral oil) was added to a solution of alcohol 1b (2.13 g, 4.73 mmol) in THF (20 mL) in one portion at 0° C. The solution was stirred for 30 minutes and carbon disulfide (0.853 mL, 14.19 mmol) was added. After 30 minutes, methyl iodide (0.444 mL, 7.10 mmol) was added slowly and the solution was stirred at rt for 40 minutes. The reaction was quenched with NH₄Cl and extracted with CH₂Cl₂. The organic layer was washed with water, brine and evaporated. Chromatographic purification (hexane/EA from 100/0 to 90/10) afforded 2b (2.35 g, 92%) as a yellow oil. IR (neat): 2927, 1441, 1362, 1236, 1207, 1144, 1063 cm⁻¹. ¹H NMR (400 MHz, CDCl₃) δ 5.88-5.75 (m, 2H), 5.17 (d, J=6.4 Hz, 2H), 3.30 (d, J=7.3 Hz, 2H), 2.71 (m, 2H), 2.56 (s, 3H), 2.44-2.30 (m, 2H). ¹³C NMR (100 MHz, CDCl₃) δ 216.0, 131.6, 125.7, 68.5, 32.2 (t, J_{C-F}=22.0 Hz), 29.2, 22.3, 19.4. ¹⁹F NMR (282 MHz, CDCl₃) δ −8.4, −42.0, −49.5, −50.5, −51.0, −53.8.

Dithiocarbonic acid S-methyl ester S-[1-(3,3,4,4,5,5,6,6,7,7,8,8,8-tridecafluoro-octylsulfanylmethyl)-allyl]ester (3b). A solution of 2b (2.23 g, 4.12 mmol) in toluene (21 mL) was heated at reflux for 4 hours. Evaporation of the reaction mixture afforded 3b (2.23 g, quant) as a colorless oil. IR (neat): 2931, 1643, 1238, 1205, 1144 cm⁻¹. ¹H NMR (400 MHz, CDCl₃) δ 5.81 (ddd, J=17.2, 10.1, 8.3 Hz, 1H), 5.35 (d, J=17.2 Hz, 1H), 5.27 (d, J=10.1 Hz, 1H), 4.36 (dt, J=8.3, 5.2 Hz, 1H), 2.96 (dd, J=13.5, 5.2 Hz, 1H), 2.84 (dd, J=13.5, 8.9 Hz, 1H), 2.88-2.76 (m, 2H), 2.43 (s, 3H), 2.50-2.30 (m, 2H). ¹³C NMR (100 MHz, CDCl₃) δ 189.1, 134.7, 119.3, 48.0, 37.2, 32.3 (t, J_{C-F}=22.0 Hz), 23.6, 13.4. ¹⁹F NMR (282 MHz, CDCl₃) δ −8.3, −41.9, −49.5, −50.6, −51.0, −53.7. ESIHRMS: calc. for C₁₄H₁₄F1₁₃O₂S₃ [M+H]⁺ 556.99428, found 556.99448 and calc. for C₁₄H₁₃F₁₃O₂S₃Na [M+Na]⁺ 578.97626, found 578.97645.

2-[1-(3,3,4,4,5,5,6,6,7,7,8,8,8-Tridecafluoro-octylsulfanylmethyl)-allyldisulfanyl]-benzothiazole (8). A solution of 3b (540 mg, 1.0 mmol) and ethanolamine (0.365 mL, 6.0 mmol) in THF (2 mL) was stirred at rt for 3 hours. The solution was degassed by sparging with argon (10 minutes) and was then added dropwise to a well stirred suspension of 2,2′-dithiobis(benzothiazole) (665 mg, 2.0 mmol) in chloroform (12 mL). The addition was performed over a period of 15 min and the reaction mixture was stirred for one hour. The mixture was filtered through Celite, and the filtrate was washed with water. The aqueous layer was extracted with CH₂Cl₂; the combined organic layers were washed with brine, dried over Na₂SO₄, filtered and evaporated. Chromatographic purification (hexane/Et₂O from 100/0 to 95/5) afforded 8 (95 mg, 15%) as a white solid. Mp: 47-48° C. IR (neat): 1462, 1425, 1234, 1144, 1004 cm⁻¹. ¹H NMR (400 MHz, CDCl₃) δ 7.87 (d, J=8.1 Hz, 1H), 7.80 (d, J=8.1 Hz, 1H), 7.44 (m, 1H), 7.34 (m, 1H), 5.76 (ddd, J=17.0, 9.8, 8.3 Hz, 1H), 5.34 (d, J=17.0 Hz, 1H), 5.31 (d, J=9.8 Hz, 1H), 3.82 (m, 1H), 3.07 (dd, J=13.3, 5.3 Hz, 1H), 2.95 (dd, J=13.3, 9.1 Hz, 1H), 2.81-2.77 (m, 2H), 2.44-2.30 (m, 2H). ¹³C NMR (100 MHz, CDCl₃) δ 172.2, 155.2, 136.1, 134.5, 126.6, 125.1, 122.5, 121.4, 121.1, 54.8, 36.1, 32.2 (t, J_{C-F}=22.0 Hz), 23.8. ¹⁹F NMR (282 MHz, CDCl₃) δ −8.3, −41.9, −49.5, −50.4, −50.9, −53.7. ESIHRMS: calc. for C₁₉H₁₅F₁₃NS₄ [M+H]⁺ 631.98743, found 631.98709.

O-(2E,6E)-3,7,11-trimethyldodeca-2,6,10-trienyl dimethylcarbamothioate (2c). Sodium hydride (1.12 g, 28 mmol, 60% in mineral oil) was washed with absolute ether (8 mL) and DMF was added (8 mL). Trans, trans-famesol 1c (5.01 mL, 20 mmol) in DMF (8 mL) was added slowly to the mixture, which then was stirred under argon for 1 hour. The mixture was cooled to 0° C., and dimethylthiocarbamoyl chloride (3.46 g, 28 mmol) was added in DMF (6 mL). The mixture was heated to 60° C. for 1 hour, then cooled and poured into 1% KOH (80 mL). The aqueous layer was extracted with ether. The ethereal layer was washed with brine, dried over Na₂SO₄, filtered and evaporated. Chromatographic purification (hexane/ether from 100/0 to 90/10) afforded 2c (4.965 g, 80%) as a slightly yellow oil. ¹H NMR (400 MHz, CDCl₃) δ 5.41 (m, 1H), 5.10-5.04 (m, 2H), 4.96 (d, J=7.0 Hz, 2H), 3.35 (s, 3H), 3.09 (s, 3H), 2.12-2.00 (m, 6H), 1.98-1.93 (m, 2H), 1.71 (s, 3H), 1.66 (s, 3H), 1.58 (s, 6H). ¹³C NMR (100 MHz, CDCl₃) δ 188.5, 142.3, 135.6, 131.5, 124.5, 123.9, 118.8, 68.8, 42.9, 39.9, 39.7, 38.0, 26.9, 26.4, 25.9, 17.9, 16.9, 16.3. ESIHRMS: calc. for C₁₈H₃₂NOS [M+H]⁺ 310.21991, found 310.21930.

(E)-S-3,7,11-trimethyldodeca-1,6,10-trien-3-yl dimethylcarbamothioate (3c). Neat thiocarbamate 2c (2.055 g, 6.62 mmol) was heated at 140° C. for 2.5 hours. Chromatographic purification (hexane/ether from 100/0 to 90/10) afforded 3c (1.70 g, 83%) as a colorless oil. IR (neat): 3085, 2966, 2922, 1655, 1450, 1360, 1092 cm⁻¹. ¹H NMR (400 MHz, CDCl₃) δ 6.15 (dd, J=17.4, 10.6 Hz, 1H), 5.16 (d, J=17.4 Hz, 1H), 5.13 (d, J=10.6 Hz, 1H), 5.12-5.05 (m, 2H), 2.94 (s, 6H), 2.06-1.84 (m, 8H), 1.67 (s, 3H), 1.60 (s, 3H), 1.59 (s, 3H), 1.58 (s, 3H). ¹³C NMR (100 MHz, CDCl₃) δ 167.9, 143.3, 135.6, 131.6, 124.6, 124.0, 113.6, 54.4, 40.4, 39.9, 36.7 (broad), 27.0, 26.0, 23.8, 23.5, 18.0, 16.3. ESIHRMS: calc. for C₁₈H₃₂NOS [M+H]⁺ 310.21991, found 310.31934.

(E)-3,7,11-trimethyldodeca-1,6,10-triene-3-thiol (4c). To a suspension of LiAlH₄ (250 mg) in anhydrous ether (20 mL) was added dropwise a solution of 3c (1.58 g, 5.10 mmol) in anhydrous ether (10 mL) so as to maintain reflux. The mixture was heated at reflux for 2 hour. Water (1 mL) was then added, followed by 10% NaOH (1 mL) and water (1.5 mL). The resulting white solid was removed by filtration and washed with ether. The organic layer was dried over Na₂SO₄, filtered and evaporated. Chromatographic purification (hexane) afforded 4c (1.049 g, 87%) as a colorless oil. ¹H NMR (400 MHz, CDCl₃) δ 5.97 (dd, J=17.3, 10.5 Hz, 1H), 5.10 (d, J=17.3 Hz, 1H), 5.11-5.06 (m, 2H), 4.98 (d,J=10.5 Hz, 1H), 2.10-1.95 (m, 6H), 1.69-1.65 (m, 2H), 1.68 (s, 3H), 1.60 (s, 6H), 1.48 (s, 3H). ¹³C NMR (100 MHz, CDCl₃) δ 145.9, 135.6, 131.4, 124.3, 123.6, 111.1, 48.3, 44.2, 39.6, 28.4, 26.6, 25.7, 23.9, 16.7, 16.0. EIHRMS: calc. for C₁₅H₂₆NS [M]⁺ 238.17552, found 238.17660.

2-(1,5,9-Trimethyl-1-vinyl-deca-4,8-dienyldisulfanyl)-benzothiazole (9). A solution of 4c (426 mg, 1.786 mmol) in chloroform (4.8 mL) was added, dropwise, to a well stirred suspension of 2,2′-dithiobis(benzothiazole) (713 mg, 2.14 mmol) in chloroform (27 mL). The addition was performed over a period of 15-30 min and the reaction mixture was stirred until completion. The solution was evaporated and disulfide was purified by chromatography (hexane/EA from 100/0 to 95/5) to afford 9 (600 mg, 83%) as a slightly yellow oil. IR (neat): 3065, 2964, 2924, 1728, 1460, 1427, 1375, 1005 cm⁻¹. ¹H NMR (400 MHz, CDCl₃) δ 7.84 (d, J=8.1 Hz, 1H), 7.77 (d, J=7.9 Hz, 1H), 7.41 (m, 1H), 7.31 (m, 1H), 5.84 (dd, J=17.3, 10.6 Hz, 1H), 5.14 (d, J=17.3 Hz, 1H), 5.11 (d, J=10.6 Hz, 1H), 5.12-5.05 (m, 2H), 2.10-1.96 (m, 6H), 1.83-1.78 (m, 2H), 1.68 (s, 3H), 1.59 (s, 6H), 1.48 (s, 3H). ¹³C NMR (100 MHz, CDCl₃) δ 174.3, 155.0, 140.7, 136.2, 136.0, 131.7, 126.7, 124.7, 124.4, 123.4, 122.3, 121.2, 116.1, 58.1, 39.90, 39.86, 26.8, 26.0, 23.8, 22.8, 18.0, 16.3. EIHRMS: calc. for C₂₂H₂₉NS₃ [M]⁺ 403.14621, found 403.1440.

2-(1,5,9-Trimethyl-1-vinyl-deca-4,8-diene)-sulfenoselenoic acid phenyl ester (10). A solution of thiol 4c (238 mg, 1.0 mmol) in CH₂Cl₂ was added to a solution of N-phenylselenophtalimide (362 mg, 1.2 mmol) at −78° C. The solution was stirred at −78° C. for 15 minutes and warmed up at rt. The heterogeneous solution was filtered over Celite and evaporated. Chromatographic purification (hexane/EA from 100/0 to 95/5) provided 10 (220 mg, 56%) as a yellow oil. ¹H NMR (400 MHz, CDCl₃) δ 7.61 (m, 2H), 7.29-7.19 (m, 3H), 5.76 (dd, J=17.4, 10.8 Hz, 1H), 5.08 (m, 1H), 5.04 (d, J=17.4 Hz, 1H), 5.03 (d, J=10.8 Hz, 1H), 5.00 (m, 1H), 2.05-1.92 (m, 6H), 1.73-1.68 (m, 2H), 1.69 (s, 3H), 1.60 (s, 3H), 1.56 (s, 3H), 1.36 (s, 3H). ¹³C NMR (100 MHz, CDCl₃) δ 142.5, 135.8, 131.8, 131.7, 130.2, 129.1, 127.3, 124.5, 123.8, 114.3, 55.0, 40.5, 39.9, 26.9, 26.0, 24.1, 23.8, 18.0, 16.3. EIHRMS: calc. for C₂₁H₃₀SSe [M]⁺ 394.12334, found 394.1213.

(E)-N-(tert-Butoxycarbonyl)-S-(tridec-2-enyl)-L-cysteine methyl ester (13). Following the general procedure for allylation sequence; N-(tert-butoxycarbonyl)-L-cysteine methyl ester 11 (59 mg, 0.25 mmol) was reacted with 8 (190 mg, 0.5 mmol) and Ph₂P-4-C₆H₄NMe₂ (228 mg, 0.75 mmol) to provide 13 (89 mg, 85%) as a colorless oil [chromatographic purification (hexane/EA from 100/0 to 95/5)]. IR (neat): 3384, 2923, 2854, 1749, 1718, 1498, 1365, 1169, 1055, 1014 cm⁻¹. [α]_{D}²¹+22.7 (c 0.52, CHCl₃). ¹H NMR (400 MHz, CDCl₃) δ 5.54 (ddd, J=15.2, 6.8, 6.7 Hz, 1H), 5.30-5.30 (m, 2H), 4.50 (m, 1H), 3.76 (s, 3H), 3.14-3.04 (AMX, J=13.5, 7.4, 7.3 Hz, 1H), 2.91 (dd, J=14.0, 4.8 Hz, 1H), 2.83 (dd, J=14.0, 5.7 Hz, 1H), 2.03 (q, J=6.7 Hz, 2H), 1.45 (s, 9H), 1.40-1.20 (m, 16H), 0.88 (t, J=6.5 Hz, 3H). Double bond configuration was attributed without ambiguity by ¹H homonuclear spin decoupling experiment (irradiation of allylic protons at 2.03 ppm enables the determination of J_{HC═CH}=15.2 Hz). ¹³C NMR (100 MHz, CDCl₃) δ 172.0, 155.4, 135.2, 125.3, 80.3, 53.3, 52.8, 34.6, 32.9, 32.6, 32.2, 29.9, 29.7, 29.6, 29.5, 28.6, 23.0, 14.4. ESIHRMS: calc. for C₂₂H₄₂NO₄S [M+H]⁺ 416.28291, found 416.28283 and calc. for C₂₂H₄₁NO₄SNa [M+Na]⁺ 438.26488, found 438.26467.

Tridec-2-enyl-(tetra-O-acetyl-1-thio-β-D-glucopyranoside) (18). Following the general procedure for allylation of 1-thio-β-D-glucose tetraacetate, 14 (36 mg, 0.10 mmol) was reacted with 5 (50 mg, 0.13 mmol), Et₃N (42 μL, 0.30 mmol) and PPh₃ (79 mg, 0.30 mmol) to afford 18 (40 mg, 73%, 2 diasteromers (ratio: 12/1)) as a white solid [chromatographic purification (hexane/EA from 100/0 to 80/20]. [α]_{D}²³ −15.2 (c 1, CHCl₃). ESIHRMS of the mixture: calc. for C₂₇H₄₄O₉SNa [M+Na]⁺ 567.26040, found 567.2585.

Spectral data of major diastereomer (18 M): ¹H NMR (400 MHz, CDCl₃) δ 5.56 (m, 1H), 5.40 (m, 1H), 5.21 (t, J=9.3 Hz, 1H), 5.08 (m, 2H), 4.47 (d, J=10.1 Hz, 1H), 4.24 (dd, J=12.4, 5.1 Hz, 1H), 4.13 (dd, J=12.4, 5.3 Hz, 1H), 3.63 (m, 1H), 3.35 (dd, J=13.2, 8.4 Hz, 1H), 3.19 (dd, J=13.2, 6.1 Hz, 1H), 2.09 (s, 3H), 2.05 (s, 3H), 2.03 (m, 2H), 2.02 (s, 3H), 2.01 (s, 3H), 1.39-1.10 (m, 16H), 0.88 (t, J=7.1 Hz, 3H). Double bond configuration was attributed without ambiguity by ¹H homonuclear spin decoupling experiment (irradiations of allylic protons at 2.03 ppm enable the determination of J_{HC═CH}=15.2 Hz). ¹³C NMR (100 MHz, CDCl₃) δ 170.9, 170.5, 169.7, 135.4, 124.7, 82.2, 76.0, 74.3, 70.2, 68.7, 62.5, 32.6, 32.4, 32.2, 29.9, 29.8, 29.6, 29.5, 23.0, 21.03, 20.99, 20.90, 20.86, 14.4.

Characteristic signals of minor diastereomer (18m): ¹H NMR (400 MHz, CDCl₃) δ 4.46 (d, J=10.1 Hz, 1H), 3.51 (dd, J=13.2, 9.3 Hz, 1H). ¹³C NMR (100 MHz, CDCl₃) δ 134.5, 124.3, 82.6, 70.0, 68.6, 62.4.

4-(3,3,4,4,5,5,6,6,7,7,8,8,8-tridecafluoro-octylsulfanyl)-but-2-enyl-(tetra-O-acetyl-1-thio-β-D-glucopyranoside) (19). Following the general procedure for allylation of 1-thio-β-D-glucose tetraacetate, 14 (36 mg, 0.10 mmol) was reacted with 8 (82 mg, 0.13 mmol), Et₃N (42 μL, 0.30 mmol) and PPh₃ (79 mg, 0.30 mmol) to provide 19 (60 mg, 75%, 2 diasteromers (ratio: 7.9/1)) as a slightly yellow solid [chromatographic purification (hexane/EA from 100/0 to 80/20]. [α]_{D}²³−13.3 (c 0.75, CHCl₃) ESIHRMS of the mixture: calc. for C₂₆H₂₉F₁₃O₉S₂ [M+Na]⁺ 819.09434, found 819.0930. Spectral data of major diastereomer (19M): ¹H NMR (400 MHz, CDCl₃) δ 5.59-5.56 (m, 2H), 5.20 (t, J=9.3 Hz, 1H), 5.06 (m, 2H), 4.46 (d, J=10.1 Hz, 1H), 4.24 (dd, J=12.5, 5.1 Hz, 1H), 4.13 (dd, J=12.5, 2.2 Hz, 1H), 3.67 (m, 1H), 3.39 (dd, J=13.2, 8.4 Hz, 1H), 3.24 (m, 1H), 3.17 (m, 2H), 2.72-3.66 (m, 2H), 3.42-3.29 (m, 2H), 2.08 (s, 3H), 2.04 (s, 3H), 2.02 (s, 3H), 2.00 (s, 3H). ¹³C NMR (100 MHz, CDCl₃) δ 170.9, 170.5, 169.7, 129.6, 128.7, 82.1, 76.1, 74.1, 70.0, 68.6, 62.4, 33.8, 32.1 (t, J=21.8 Hz), 31.3, 21.7, 21.0, 20.9, 20.8.

Characteristic signals of minor diastereomer (19m): ¹H NMR (400 MHz, CDCl₃) δ 5.68-5.60 (m, 2H), 5.22 (t, J=9.4 Hz, 1H), 4.44 (d, J=10.0 Hz, 1H), 3.70 (m, 1H). ¹³C NMR (100 MHz, CDCl₃) δ 129.0, 127.7, 82.2, 74.0, 69.8, 60.7.

3,7,11-trimethyl-dodeca-2,6,10-trienyl-(tetra-O-acetyl-1-thio-β-D-glucopyranoside) (20). Following the general procedure for allylation of 1-thio-β-D-glucose tetraacetate, 14 (36 mg, 0.10 mmol) was reacted with 9 (52 mg, 0.13 mmol), Et₃N (42 μL, 0.30 mmol) and PPh₃ (79 mg, 0.30 mmol) to provide 20 (40 mg, 66%, 2 diasteromers (ratio: 1.8/1)) as a white solid [chromatographic purification (hexane/EA from 100/0 to 85/15]. ¹H NMR (400 MHz, CDCl₃) δ 5.22-5.17 (m, 2×2H), 5.13-5.02 (m, 2×4H), 4.43 (d, J=10.0 Hz, 1H, dias minor), 4.42 (d, J=10.1 Hz, 1H, dias major), 4.25 (dd, J=12.4, 4.8 Hz, 1H, dias minor), 4.23 (d, J=12.4, 5.0 Hz, 1H, dias major), 4.15-4.09 (m, 2×1H), 3.65-3.60 (m, 2×1H), 3.49 (dd, J=13.0, 9.2 Hz, 1H, dias major), 3.45 (dd, J=13.0, 8.9 Hz, 1H, dias minor), 3.20 (m, 2×1H), 2.13-1.96 (m, 2×8H), 2.07 (s, 2×3H), 2.04 (s, 2×3H), 2.02 (s, 2×3H), 2.00 (s, 2×3H), 1.75 (s, 3H, dias minor), 1.67 (s, 2×3H), 1.66 (s, 3H, dias major), 1.59 (s, 2×6H). ¹³C NMR (100 MHz, CDCl₃) δ 170.9, 170.54, 170.49, 169.7, 140.8, 140.7, 136.1, 135.8, 131.7, 131.6, 124.6, 124.5, 123.9, 123.7, 120.0, 119.2, 82.7, 82.4, 76.10, 76.05, 74.2, 70.1, 68.7, 68.6, 62.5, 62.4, 40.0, 39.9, 31.9, 30.0, 27.5, 27.0, 26.94, 26.86, 26.7, 26.0, 23.7, 21.00, 20.98, 20.90, 20.86, 18.0, 16.3, 16.2. ESIHRMS of the mixture: calc. for C₂₉H₄₄O₉SNa [M+Na]⁺ 591.26040, found 591.2590.

N-(tert-Butoxycarbonyl)-S-(3,7,11-trimethyl-dodeca-2,6,10-trienyl)-L-cysteine methyl ester (21). Following the general procedure for allylation sequence, N-(tert-butoxycarbonyl)-L-cysteine methyl ester 11 (26 mg, 0.11 mmol) was reacted with 5 (88 mg, 0.22 mmol) Et₃N (77 μL, 0.55 mmol) and PPh₃ (88 mg, 0.33 mmol) to afford 21 (41 mg, 84%, 2 diasteromers (ratio: 1.87/1)) as a slightly yellow oil [chromatographic purification (hexane/EA from 100/0 to 95/5)]. ESIHRMS of the mixture of diastereomers: calc. for C₂₄H₄₁NO₄SNa [M+Na]⁺ 462.26543, found 462.2656. Pure samples of major and minor diastereomers were obtained after additional purification by normal phase HPLC (Hexane/EA gradient).

Spectral data of major diastereomer (21M): ¹H NMR (500 MHz, CDCl₃) δ 5.30 (d, J=7.5 Hz, 1H), 5.21 (dt, J=7.7, 1.0 Hz, 1H), 5.10-5.07 (m, 2H), 4.52 (m, 1H), 3.76 (s, 3H), 3.20 (dd, J=13.1, 8.0 Hz, 1H), 3.15 (dd, J=13.1, 7.5 Hz, 1H), 2.91 (dd, J=13.8, 4.9 Hz, 1H), 2.85 (dd, J=13.8, 5.7 Hz, 1H), 2.13-2.04 (m, 6H), 1.99-1.95 (m, 2H), 1.68 (s, 3H), 1.67 (s, 3H), 1.60 (s, 6H), 1.45 (s, 9H). ¹³C NMR (125 MHz, CDCl₃) δ 172.0, 155.1, 140.3, 135.7, 131.6, 124.6, 124.0, 120.0, 80.4, 53.5, 52.8, 40.0, 39.9, 34.0, 30.3, 28.6, 27.0, 26.7, 26.0, 18.0, 16.4, 16.3.

Spectral data of minor diastereomer (21m): ¹H NMR (400 MHz, CDCl₃) δ 5.31 d, J=7.7 Hz, 1H), 5.22(dt, J=67, 1.0 Hz, 1H), 5.09 (m, 2H), 4.52 (m, 1H), 3.75 (s, 3H), 3.16 (m, 2H), 2.91 (m, 2H), 2.11-1.95 (m, 8H), 1.74 (s, 3H), 1.68 (s, 3H), 1.60 (s, 6H), 1.45 (s, 9H). ¹³C NMR (100 MHz, CDCl₃) δ 172.0, 155.5, 140.3, 136.0, 131.7, 124.6, 123.9, 120.7, 80.4, 53.6, 52.8, 40.4, 34.3, 32.1, 30.4, 28.6, 27.0, 26.8, 26.0, 23.7, 18.0, 16.3.

The double bond configuration of 21M (E) and 21m (Z) have been attributed by ¹³C γ effect. Methyl in a sterically crowded environment (such as the methylene carbon of 21M at 16.4 ppm) are upfield of methyl that are not (such as the methylene carbon of 21m at 23.7 ppm).

(E)-N-(tert-Butoxycarbonyl)-S-(tridec-2-enyl)-glutathione dimethyl ester (22). Following the general procedure for allylation sequence, the glutathione derivative 25 (65 mg, 0.149 mmol) was reacted with 17 (110 mg, 0.298 mmol) and PPh₃ (118 mg, 0.447 mmol) to provide 22 (65 mg, 70%) as a slightly yellow gum [chromatographic purification (hexane/EA from 50/50 to 40/60)]. IR (neat): 3302, 2926, 1747, 1718, 1647, 1527, 1211, 1173 cm⁻¹. [α]_{D}²¹−8.1 (c 0.65, CDCl₃). ¹H NMR (400 MHz, CDCl₃) δ 7.04 (m, 1H), 6.75 (bd, J=6.9 Hz, 1H), 5.60 (m, 1H), 5.38 (m, 1H), 5.32 (bd, J=7.9 Hz, 1H), 4.57 (q, J=6.8 Hz, 1H), 4.39 (m, 1H), 4.09 (dd, J=18.1, 5.6 Hz, 1H), 4.00 (dd, J=18.1, 5.3 Hz, 1H), 3.75 (s, 3H), 3.74 (s, 3H), 3.14 (d, J=7.3, 2H), 2.87 (m, 2H), 2.38-2.32 (m, 2H), 2.18 (m, 1H), 2.08-1.91 (m, 3H), 1.43 (s, 9H), 1.40-1.24 (m, 16H), 0.87 (t, J=7.0 Hz, 3H). Double bond configuration was attributed without ambiguity by ¹H homonuclear spin decoupling experiment (irradiation of allylic protons at 2.08-1.91 ppm enables the determination of J_{HC═CH}=15.2 Hz). ¹³C NMR (125 MHz, CDCl₃) δ 173.1, 172.4, 171.0, 170.2, 155.9, 135.2, 125.3, 80.4, 53.0, 52.8, 52.6, 52.5, 41.5, 34.5, 32.6, 32.4, 32.2, 29.9, 29.7, 29.6, 29.53, 29.48, 28.8, 28.5, 22.9, 14.4. ESIHRMS: calc. for C₃₀H₅₄N₃O₈S [M+H]⁺ 616.36261, found 616.36251 and calc. for C₃₀H₅₃N₃O₈SNa [M+Na]⁺ 638.34459, found 638.34436.

N-(tert-Butoxycarbonyl)-S-(4-(3,3,4,4,5,5,6,6,7,7,8,8,8-tridecafluoro-octylsulfanyl)-but-2-enyl)-glutathione dimethyl ester (23). Following the general procedure for allylation sequence, the glutathione derivative 15 (22 mg, 0.05 mmol) was reacted with 8 (63 mg, 0.1 mmol) and PPh₃ (39 mg, 0.15 mmol) to afford 23 (33 mg, 75%) as a colorless gum [chromatographic purification (CHCl₃/MeOH from 100/0 to 98/2)]. IR (neat): 3307, 2980, 1749, 1653, 1541, 1236 cm⁻¹. [α]_{D}²¹−4.0 (c 0.2, CHCl₃). ¹H NMR (400 MHz, CDCl₃) δ 7.06 (bs, 1H), 6.81 (bd, J=6.5 Hz, 1H), 5.68-5.52 (m, 2H), 5.31 (bs, 1H), 4.58 (m, 1H), 4.38 (bs, 1H), 4.10-3.96 (m, 2H), 3.744 (s, 3H), 3.737 (s, 3H), 3.22-3.16 (m, 4H), 2.91-2.81 (m, 2H), 2.70-2.65 (m, 2H), 2.41-2.29 (m, 4H), 2.18 (m, 1H), 1.95 (m, 1H), 1.43 (s, 9H). ¹H NMR (400 MHz, MeOD) δ 5.68-5.45 (m, 2H), 4.55 (dd, J=8.0, 5.9 Hz, 1H), 4.15 (dd, J=8.8, 4.8 Hz, 1H), 3.95 (s, 2H), 3.72 (s, 3H), 3.71 (s, 3H), 3.22 (d, J=6.2 Hz, 2H), 3.19 (d, J=6.3 Hz, 2H), 2.95 (dd, J=14.0, 5.7 Hz, 1H), 2.75-2.65 (m, 3H), 2.52-2.35 (m, 4H), 2.12 (m, 1H), 1.90 (m, 1H), 1.44 (s, 9H). Double bond configuration was attributed without ambiguity by ¹H homonuclear spin decoupling experiment (simultaneous irradiation of allylic protons at 3.22 and 3.19 ppm enables the determination of J_{HC═CH}=15.1 Hz). ¹³C NMR (100 MHz, MeOD) δ 173.4, 173.1, 171.9, 170.1, 156.6, 129.1, 128.9, 79.2, 53.1, 52.4, 51.3, 51.2, 40.4, 32.6, 32.5, 31.9, 31.43, 31.37 (t, J_{C-F}=21.9 Hz), 27.3, 27.0, 20.6. ¹⁹F NMR (282 MHz, CDCl₃) δ −8.3, −41.9, −49.5, −50.5, −51.0, −53.7. ESIHRMS: calc. for C₂₉H₃₈F₁₃N₃O₈S₂Na [M+Na]⁺ 890.1785, found 890.17908.

N-(tert-Butoxycarbonyl)-S-(3,7,11-trimethyl-dodeca-2,6,10-trienyl)-glutathione dimethyl ester (24). Following the general procedure for allylation sequence, the glutathione derivative 15 (48 mg, 0.11 mmol) was reacted with 9 (88 mg, 0.22 mmol), Et₃N (77 μL, 0.55 mmol) and PPh₃ (87 mg, 0.33 mmol) to provide 24 (50 mg, 70%, 2 diasteromers (ratio: 1.6/1)) as a yellow oil ESILRMS of the mixture of diastereomers: calc. for C₃₂H₅₃N₃O₈SNa [M+Na]⁺ 678.8 (15). [Chromatographic purification (hexane/EA from 100/0 to 30/70) to afford 60 mg of 24 (contaminated by a phosphorous byproduct), followed by normal phase HPLC purification (Hexane/EA gradient)].

Spectral data of major diastereomer (24M): ¹H NMR (400 MHz, CDCl₃) δ 7.05 (m, 1H), 6.75 (m, 1H), 5.29 (m, 1H), 5.25 (dd, J=7.7, 7.3 Hz, 1H), 5.09 (m, 2H), 4.56 (m, 1H), 4.40 (m, 1H), 4.12-3.97 (m, 2H), 3.75 (s, 3H), 3.74 (s, 3H), 3.23 (m, 2H), 2.92 (dd, J=14.0, 6.5 Hz, 1H), 2.85 (dd, J=14.0, 6.6 Hz, 1H), 2.40-2.32 (m, 2H), 2.19 (m, 1H), 2.12-1.93 (m, 9H), 1.68 (s, 6H), 1.60 (s, 3H), 1.59 (s, 3H), 1.44 (s, 9H). ¹H NMR (400 MHz, MeOD) δ 5.23 (t, J=7.7 Hz, 1H), 5.13-5.07 (m, 2H), 4.55 (dd, J=8.6, 5.5 Hz, 1H), 4.16 (dd,J=9.0, 5.1 Hz, 1H), 3.95 (s, 2H), 3.72 (s, 3H), 3.71 (s, 3H), 3.20 (m, 2H), 2.96 (dd, J=13.8, 5.5 Hz, 1H), 2.67 (dd, J=13.8, 8.9 Hz, 1H), 2.37 (t, J=7.5, 2H), 2.18-2.01 (m, 7H), 2.00-1.88 (m, 3H), 1.69 (s, 3H), 1.67 (s, 3H), 1.60 (s, 3H), 1.44 (s, 9H). ¹³C NMR (100 MHz, MeOD) δ 173.4, 173.0, 172.1, 170.1, 156.9, 139.6, 134.8, 130.7, 124.0, 123.7, 120.0, 79.2, 53.1, 52.7, 51.3, 51.2, 40.5, 39.4, 39.3, 32.4, 31.5, 28.9, 27.3, 27.1, 26.4, 26.0, 24.5, 16.4, 14.9, 14.7.

Spectral data of minor diastereomer (24m): ¹H NMR (400 MHz, CDCl₃) δ 7.02 (m, 1H), 6.72 (m, 1H), 5.30 (m, 1H), 5.25 (dd, J=7.8, 7.2 Hz, 1H), 5.14-5.06 (m, 2H), 4.56 (m, 1H), 4.39 (m, 1H), 4.11-3.97 (m, 2H), 3.75 (s, 3H), 3.74 (s, 3H), 3.21 (m, 2H), 2.95 (dd, J=13.9, 6.4 Hz, 1H), 2.85 (dd, J=13.9, 6.5 Hz, 1H), 2.40-2.32 (m, 2H), 2.20 (m, 1H), 2.12-1.94 (m, 9H), 1.74 (s, 3H), 1.68 (s, 3H), 1.60 (s, 6H), 1.44 (s, 9H). ¹H NMR (400 MHz, MeOD) δ 5.25 (dd, J=8.0, 1.6 Hz, 1H), 5.14 (m, 1H), 5.09 (m, 1H), 4.55 (dd, J=8.5, 5.6 Hz, 1H), 4.15 (dd, J=9.0, 5.1 Hz, 1H), 3.95 (s, 2H), 3.72 (s, 3H), 3.71 (s, 3H), 3.20 (d, J=7.8 Hz, 2H), 2.97 (dd, J=13.8, 5.6 Hz, 1H), 2.69 (dd, J=13.8, 8.7 Hz, 1H), 2.37 (t, J=7.6, 2H), 2.18-2.04 (m, 7H), 1.99-1.89 (m, 3H), 1.74 (s, 3H), 1.67 (s, 3H), 1.61 (s, 3H), 1.60 (s, 3H), 1.44 (s, 9H). ¹³C NMR (100 MHz, MeOD) δ 173.4, 173.0, 172.1, 170.1, 156.4, 139.2, 135.0, 130.7, 124.0, 123.7, 120.6, 79.2, 53.1, 52.7, 51.3, 51.2, 40.5, 39.4, 32.8, 31.5, 31.3, 29.0, 27.3, 27.0, 26.3, 26.2, 24.5, 22.2, 16.4, 14.7.

The double bond configuration of 24M (E) and 24m (Z) have been assigned by ¹³C γ effect. Methyl in a sterically crowded environment (such as the methylene carbon of 24M at 14.9 ppm) are upfield of a methyl that are not (such as the methylene carbon of 24m at 22.2 ppm).

(E)-N-(tert-Butoxycarbonyl)-S-(tridec-2-enyl)-L-cysteinyl-L-alanyl-L-tryptophane methyl ester (25). Following the general procedure for allylation sequence, the tripeptide 16 (67 mg, 0.137 mmol) was reacted with 6 (89 mg, 0.275 mmol) and PPh₃ (108 mg, 0.411 mmol) to provide 25 (73 mg, 80%) as a vitreous solid [chromatographic purification (CHCl₃/MeOH from 100/0 to 99/1]. IR (neat): 3394, 3303, 3059, 2926, 2852, 1691, 1649, 1521, 1170 cm⁻¹. [Ε]_{D}²¹+17.7 (c 0.45, CHCl₃). ¹H NMR (400 MHz, CDCl₃) δ 8.50 (bs, 1H), 7.49 (d, J=7.8 Hz, 1H), 7.33 (d, J=8.1 Hz, 1H), 7.15 (t,J=7.1 Hz, 1H), 7.09 (t,J=7.1 Hz, 1H), 6.96 (s, 1H), 6.82-6.80 (m, 2H), 5.57 (ddd, J=15.2, 6.8, 6.7 Hz, 1H), 5.38-5.30 (m, 2H), 4.88 (q, J=5.5 Hz, 1H), 4.51 (m, 1H), 4.20 (bs, 1H), 3.68 (s, 3H), 3.32 (dd, J=14.9, 5.3, 1H), 3.27 (dd, J=14.9, 5.8, 1H), 3.08 (m, 1H), 2.82 (dd, J=13.9, 5.4, 1H), 2.68 (dd, J=13.9, 6.5, 1H), 2.01 (q, J=6.8, 2H), 1.47 (s, 9H), 1.31 (d, J=7.0 Hz, 3H), 1.35-1.24 (m, 16H), 0.88 (t, J=7.0 Hz, 3H). Double bond configuration was attributed without ambiguity by ¹H homonuclear spin decoupling experiment (irradiation of allylic protons at 2.01 ppm enables the determination of J_{HC═CH}=15.2 Hz). ¹³C NMR (125 MHz, CDCl₃) δ 172.2, 171.5, 170.8, 155.8, 136.3, 135.5, 127.8, 125.2, 123.5, 122.4, 119.8, 118.7, 111.6, 109.8, 81.0, 54.0, 53.1, 52.7, 49.2, 34.6, 33.3, 32.6, 32.2, 29.9, 29.7, 29.6, 29.54, 29.51, 28.6, 27.7, 23.0, 18.2, 14.4. ESIHRMS: calc. for C₃₆H₅₇N₄O₆S [M+H]⁺ 673.39933, found 673.39946 and calc. for C₃₅H₅₆N₄O₆SNa [M+Na]⁺ 695.38131, found 695.38153.

N-(tert-Butoxycarbonyl)-S-(4-(3,3,4,4,5,5,6,6,7,7,8,8,8-tridecafluoro-octylsulfanyl)-but-2-enyl)-L-cysteinyl-L-alanyl-L-tryptophane methyl ester (26). Following the general procedure for allylation sequence, the tripeptide 16 (37 mg, 0.075 mmol) was reacted with 8 (95 mg, 0.15 mmol) and PPh₃ (59 mg, 0.225 mmol) to afford 26 (55 mg, 79%) as a vitreous solid [chromatographic purification (CHCl₃/MeOH from 100/0 to 99/1]. [α]_{D}²¹+16.5 (c 0.2, CHCl₃). ¹H NMR (400 MHz, CDCl₃) δ 8.44 (bs, 1H), 7.49 (d, J=7.8 Hz, 1H), 7.33 (d, J=7.9 Hz, 1H), 7.16 (t, J=7.1 Hz, 1H), 7.09 (t, J=7.1 Hz, 1H), 6.97 (bs, 1H), 6.80 (bd, J=7.4 Hz, 1H), 6.71 (bd, J=6.8 Hz, 1H), 5.62-5.46 (m, 2H), 5.32 (d, J=7.2 Hz, 1H), 4.89 (quint, J=7.2 Hz, 1H), 4.19 (bs, 1H), 3.69 (s, 3H), 3.34 (dd, J=14.8, 5.3, 1H), 3.28 (dd, J=14.8, 5.6, 1H), 3.14 (d, J=6.8, 2H), 3.13-3.04 (m, 2H), 2.80 (dd, J=14.0, 5.7, 1H), 2.72-2.64 (m, 3H), 2.41-2.27 (m, 2H), 1.47 (s, 9H), 1.32 (d, J=7.2 Hz, 3H). Double bond configuration was attributed without ambiguity by ¹H homonuclear spin decoupling experiment (irradiations of allylic protons at 3.14 or 3.13-3.01 ppm enable the determination of J_{HC═CH}=15.1 Hz). ¹³C NMR (125 MHz, CDCl₃) δ 172.2, 171.1, 170.6, 155.8, 136.3, 129.5, 129.2, 127.8, 123.4, 122.4, 119.9, 118.7, 111.6, 109.9, 81.1, 54.0, 53.1, 52.7,49.2, 33.8, 33.4, 32.0 (t, J_{C-F}=21.9 Hz), 28.6, 27.7, 21.6, 18.2. ¹⁹F NMR (282 MHz, CDCl₃) δ −8.3, −41.9, −49.5, −50.5, −51.0, −53.7. ESIHRMS: calc. for C₁₅H₄₁F1₁₃N₄O₆S₂Na [M+Na]⁺ 947.21524, found 947.21587.

N-(tert-Butoxycarbonyl)-S-(3,7,11-trimethyl-dodeca-2,6,10-trienyl)-L-cysteinyl-L-alanyl-L-tryptophane methyl ester (27). Following the general procedure for allylation sequence, the tripeptide 16 (59 mg, 0.11 mmol) was reacted with 9 (88 mg, 0.22 mmol), Et₃N (77 μL, 0.55 mmol) and PPh₃ (87 mg, 0.33 mmol) to provide 27 (66 mg, 79%, 2 diasteromers (ratio: 1.6/1)) as a colorless solid [chromatographic purification (hexane/EA from 100/0 to 30/70)]. IR (neat): 3396, 3307, 3055, 2974, 2927, 2854, 1726, 1691, 1649, 1523, 1169 cm⁻¹. [α]_{D}²¹+16.1 (c 1, CHCl₃) ¹H NMR (400 MHz, CDCl₃) δ 8.60 (s, 2×1H), 7.48 (d, J=7.8 Hz, 2×1H), 7.31 (d, J=8.0 Hz, 2×1H), 7.15-7.06 (m, 2×2H), 6.95 (s, 2×1H), 6.89 (bs, 2×1H), 5.40 (d, J=7.3 Hz, 2×1H), 5.20 (m, 2×1H), 5.11-5.06 (m, 2×2H), 4.87 (m, 2×1H), 4.50-4.41 (m, 2×1H), 4.27-4.15 (m, 2×1H), 3.67 (s, 2×3H), 3.28 (m, 2×2H), 3.12 (d, J=7.8 Hz, 2×2H), 2.83 (m, 2×1H), 2.71 (dd, J=13.8, 6.2 Hz, 2×1H), 2.10-1.95 (m, 2×8H), 1.73 (s, 3H, dias major), 1.67 (s, 2×3H), 1.65 (s, 3H, dias minor), 1.59 (s, 2×6H), 1.47 (s, 2×9H), 1.30 (d, J=7.0 Hz, 2×3H). ¹³C NMR (100 MHz, CDCl₃) δ 172.3, 171.6, 170.8, 155.9, 140.4, 136.3, 135.9, 135.6, 131.62, 131.58, 127.7, 124.6, 124.0, 123.8, 123.5, 122.3, 120.7, 119.8, 119.7, 118.6, 111.6, 109.7, 81.0, 54.2, 53.1, 52.7, 49.1, 40.0, 39.9, 34.2, 34.0, 33.6, 32.1, 30.22, 30.18, 27.7, 27.0, 26.8, 26.7, 26.0, 23.7, 18.3, 18.0, 16.4, 16.3. ESIHRMS: calc. for C₃₈H₅₇N₄O₆S [M+H]⁺ 697.39933, found 697.40031 and calc. for C₃₈H₅₆N₄O₆SNa [M+Na]⁺ 719.38131, found 719.38170.

S-(tridec-2-enyl)-glutathione (28). Glutathione 17 (10 mg, 0.03 mmol) was dissolved in 0.7 mL Tris buffer (0.2 M, pH 8) to which disulfide 6 (36 mg, 0.09 mmol) dissolved in 0.7 mL CH₃CN/THF (1:1) was added, and the mixture was stirred at room temperature for 10 h. After 10 h, a few drops of trifluoroacetic acid was added to obtain a homogenous solution followed by the addition of PPh₃ (43 mg, 0.16 mmol). The reaction was stirred at room temperature for additional 24 h and purified by reversed-phase HPLC using a gradient of 50% A/50% B to 100% A, developed over 50 min (A, 0.1% TFA/CH₃CN; B, 0.1% TFA/H₂O; column. Varian Microsorb C₁₈250×21.4 mm; flow rate. 8 mL/min; UV detection. 215 nm). Lyophillization of the fraction eluting at 28 min afforded lipidated glutathione 28 (11 mg, 70%) as a white powder. Mp 86-88° C. [α]_{D}²³−27.1 (c 0.6, MeOH). ¹H-NMR (400 MHz, CD₃OD) δ 5.63-5.58 (m, 1H), 5.42-5.36 (m, 1H), 4.57-4.51 (m, 1H), 4.03 (bt, J=4.8 Hz, 1H), 3.94 (d, J=15.2 Hz, 2H), 3.13 (d, J=5.3 Hz, 2H), 2.94 (dt, J=11.6 Hz, J=4 Hz, 1H), 2.68 (dd, J=11.2 Hz, J=7.2 Hz, 1H), 2.57 (t, J=5.6 Hz, 2H), 2.25-2.15 (m, 2H), 2.04 (dd, J=11.2 Hz, J=6.0 Hz, 2H), 1.37-1.28 (m, 16 H), 0.89 (t, J=5.6 Hz, 3H). Double bond configuration was attributed without ambiguity by ¹H homonuclear spin decoupling experiment (irradiation of allylic protons at 2.04 ppm enabled the determination of J_{HC═CH}=15.0 Hz). ¹³C NMR (100 MHz, CD₃OD) δ 172.9, 171.9, 170.1, 134.1, 125.5, 52.6, 52.1, 40.4, 33.3, 31.9, 31.7, 31.6, 30.9, 29.3, 29.2, 29.1, 29.0, 25.7, 22.3, 13.1. ESIHRMS: calc. for C₂₃H₄₁N₃O₆S₁ [M+H]⁺ 488.2794, found 488.2787.

In summary, the present invention provides a new and convenient, functionalization method of thiols, particularly thiols of complex molecules such as amino acids, peptides, and carbohydrates. The methods of the invention combine the use of stable and easily prepared disulfides as sulfenylating agents with a phosphine-promoted desulfurative allylic rearrangement. As demonstrated by prenylation of unprotected glutathione, this method is useful for the ligation to peptides and protein-based thiols, which have value as intermediates for the preparation of pharmaceuticals and as tools for studying the biological role of lipidated peptides and carbohydrates. The facile synthesis of the allyl hetereoaryl disulfides coupled with the applicability of the method to native peptides renders the method highly competitive with other routes to cysteine functionalized peptides, all of which require the use of electrophilic reagents, or the prior derivatization of the peptide

The use of the terms “a” and “an” and “the” and similar references in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for the preparation of an allylic sulfide comprising contacting an activated allylic sulfenyl compound of Formula (I) with a thiol of Formula (II) for a period of time sufficient to form an intermediate of Formula (III), and contacting the intermediate of Formula (III) with a thiophilic agent, in a polar solvent, to induce a [2,3]-sigmatropic rearrangement therein and thereby form an allylic sulfide of Formula (IV), with concomitant loss of a sulfur atom to the thiophilic agent: 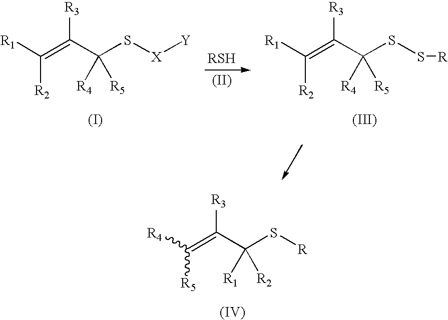
wherein X is S or SO₂; Y is an aryl group, a substituted-aryl group, a heteroaryl group, or a substituted heteroaryl group; R¹, R², R³, R⁴, and R⁵ are each independently H, a hydrocarbon moiety or a substituted hydrocarbon moiety; and R is an organic moiety.

2. The method of claim 1 wherein the organic moiety, R, is an alkyl group, a substituted-alkyl group, an aryl group, a substituted-aryl group, an amino acid, a carbohydrate, a peptide, a nucleic acid, or a group consisting of two or more of the foregoing bound together.

3. The method of claim 1 wherein X is S and Y is selected from the group consisting of an aryl group, a substituted aryl group, heteroaryl group, and a substituted heteroaryl group.

4. The method of claim 1 wherein X is S and Y is selected from the group consisting of a phenyl group, a substituted-phenyl group, a pyridyl group, a substituted pyridyl group, and a benzothiazolyl group.

5. The method of claim 1 wherein the solvent is selected from the group consisting of acetonitrile, tetrahydrofuran, a C₁-C₃ alcohol, water, an aqueous buffer, and a combination of two or more of the foregoing solvents.

6. The method of claim 1 wherein the thiophilic agent is a phosphine reagent.

7. The method of claim 6 wherein the phosphine reagent is triphenylphosphine.

8. The method of claim 6 wherein the phosphine reagent is a polymer-bound phosphine.

9. The method of claim 1 wherein the thiol of Formula (II) is a thio-substituted amino acid.

10. The method of claim 1 wherein the thiol of Formula (II) is a thio-substituted peptide.

11. The method of claim 1 wherein the thiol of Formula (II) is a thio-substituted carbohydrate.

12. A method for the preparation of an allylic sulfide comprising contacting an activated allylic sulfenyl compound of Formula (I) with a thiol of Formula (II) for a period of time sufficient to form an intermediate of Formula (III), and contacting the intermediate of Formula (III) with a thiophilic agent, in a polar solvent, to induce a [2,3]-sigmatropic rearrangement therein and thereby form an allylic sulfide of Formula (IV), with concomitant loss of a sulfur atom to the thiophilic agent: 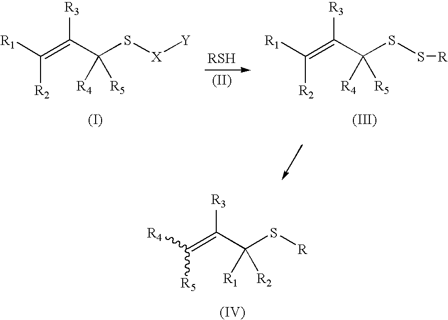
wherein X is S; Y is selected from the group consisting of a phenyl group, a substituted-phenyl group, a pyridyl group, a substituted pyridyl group, and a benzothiazolyl group; R¹, R², R³, R⁴, and R⁵ are each independently H or a hydrocarbon moiety; and R is an organic moiety selected from the group consisting of an amino acid, a peptide, and a carbohydrate.

13. The method of claim 12 wherein R¹, R², R³, R⁴, and R⁵ are each independently H, an alkyl group, or a substituted alkyl group.

14. The method of claim 12 wherein the solvent is selected from the group consisting of acetonitrile, tetrahydrofuran, a C₁-C₃ alcohol, water, an aqueous buffer, and a combination of two or more of the foregoing solvents.

15. The method of claim 12 wherein the thiophilic agent is a phosphine reagent.

16. The method of claim 15 wherein the phosphine reagent is triphenylphosphine.

17. The method of claim 15 wherein the phosphine reagent is a polymer-bound phosphine.

18. The method of claim 1 wherein the thiol of Formula (II) is a thio-substituted amino acid.

19. The method of claim 1 wherein the thiol of Formula (II) is a thio-substituted peptide.

20. The method of claim 1 wherein the thiol of Formula (II) is a thio-substituted carbohydrate.
